(19) United States
(12) Patent Application Publication
(10) Pub. No.: US 2017/0365151 A1
(43) Pub. Date: Dec. 21, 2017

(54) DEVICE FOR NOTIFICATION OF GAS CONDITIONS SYSTEM AND METHOD

(71) Applicant: MICHAEL BURLESON, SAHUARITA, AZ (US)

(72) Inventor: MICHAEL BURLESON, SAHUARITA, AZ (US)

(21) Appl. No.: 15/624,232

(22) Filed: Jun. 15, 2017

Related U.S. Application Data
(60) Provisional application No. 62/351,863, filed on Jun. 17, 2016.

(51) Int. Cl. G08B 21/14; G01N 33/00; G08B 21/16; G08B 25/10

(52) U.S. Cl. CPC G08B 21/14; G08B 25/10; G01N 33/0004; G08B 21/16

(57) ABSTRACT

A device for notification of gas conditions including means for detecting a gas condition, means for communicating the gas condition to a user, means for communicating gas condition to a third party remote from the user, means for determining a location of the device, at least one rechargeable battery, a memory configured for recording the gas condition and the location of the device, and an attachment fastener configured to removably affix the device to the user.

### CROSS-REFERENCE TO RELATED APPLICATION

The present application is related to and claims priority from prior provisional application Ser. No. 62/351,863, filed Jun. 17, 2016 which application is incorporated herein by reference in its entirety.

### BACKGROUND OF THE INVENTION

The following includes information that may be useful in understanding the present disclosure. It is not an admission that any of the information provided herein is prior art, or material, to the presently described or claimed inventions, or that any publication or document that is specifically or implicitly referenced is prior art.

### 1. Field of the Invention

The present disclosure relates generally to the field of gas monitoring and more specifically relates to a device for notification of gas conditions.

### 2. Description of Related Art

The extraction of oil and gas (i.e., fossil fuel) can sometimes produce various air-borne gasses which may be toxic to humans. Additional industries can also produce gasses which may be dangerous. Some of such gases may be explosive, corrosive, and/or otherwise dangerous or deadly in a variety of different ways. Often times, the skilled workers in the oil and gas industry utilize devices that can sense the presence of such gasses. Some such devices utilize electronic or similar type detecting means to determine the presence of such gasses and may also alert the skilled worker of such a presence. One such device can be referred to as a gas detector.

In general, a gas detector (or gas monitor) is a device that detects the presence of particular gases in an area. In some cases, the detection may be used as a safety precaution. A gas detector can sound an alarm to operators in the area where the leak is occurring, giving them the opportunity to leave. This type of device is important because there are many gases that can be harmful to organic life, such as humans or animals. Some gas monitors may be semi-permanently affixed to a jobsite. In other cases, gas monitors may be affixed to workers to alert the individual to the presence of a potentially dangerous gas.

Oil and gas sites use personal monitoring devices to detect the presence of hydrogen sulfide gas. However, these devices only alert the person using the device of the unsafe environment and not others on the job site or emergency personnel. This individual then must separately reach out to each department to report the detection of gas. In the time that this process takes, the situation can become dire. A suitable solution is desired.

Several attempts have been made to solve the above-mentioned problems such as those found in U.S. Pat. No. 6,252,510 to Dungan; U.S. Pat. No. 8,400,317 to Johnson, Jr. et al.; as well as U.S. Pat. No. 6,856,253, U.S. Pat. No. 7,463,160, U.S. Pat. No. 6,954,143, and U.S. Pat. No. 8,836,520, all to Crook. This art is representative of gas monitoring. However, none of the above references, taken either singly or in combination, is seen to describe the invention as claimed. Thus, a need exists for a reliable device for notification of gas conditions to avoid the above-mentioned problems.

### BRIEF SUMMARY OF THE INVENTION

In view of the foregoing disadvantages inherent in the known gas monitoring art, the present disclosure provides a novel device for notification of gas conditions such as gas leaks. The general purpose of the present disclosure, which will be described subsequently in greater detail, is to provide a device to alert a user, as well as one or more third parties remote from the user, to the presence of dangerous gasses, while providing the user with the ability to also manually alert the third parties of certain conditions. The device can also alert other users, with similar devices, in proximity of the user.

A device for notification of gas conditions is disclosed herein, in a preferred embodiment, including means for detecting a gas condition, means for communicating the gas condition to a user, means for communicating the gas condition to a third party remote from the user, means for determining a location of the device, at least one rechargeable battery, a memory configured for recording the gas condition and the location of the device, and an attachment fastener configured to removably affix the device to the user.

Preferably, the means for detecting the gas condition including a sensor configured to detect hydrogen-sulfide gas, an oxygen sensor, a sensor to include the determination of an oxygen-rich condition and oxygen-deficient condition, as well as a sensor configured to detect carbon monoxide. The means for detecting the aforementioned gasses may include one sensor dedicated to detect all gasses, or a plurality of sensors each configured to detect all gasses, or a combination of sensors.

Preferably, the means for communicating the gas leak or gas condition to the user is configured to communicate a plurality of severities of the gas condition to the user and others. The plurality of severities of the gas condition may include a caution-level of severity, a danger-level of severity, and a catastrophic-level of severity. The plurality of severities of the gas condition may also be manually alertable, by the user, to the third party remote from the user, as well as additional users with similar devices on-site.

Also, the means for communicating the gas condition (e.g., a gas leak, the presence of dangerous or undesirable gas conditions, the deficiency of healthy gas conditions, predefined gas conditions particular to the use/application of the device) to the user also includes a display configured to visually communicate the gas condition to the user. Additionally, the means for communicating the gas condition to the user also includes a speaker configured to aurally communicate the gas condition to the user.

Also, the means for communicating the gas condition to the user is configured to communicate to a plurality of devices (and therefore additional users) to the presence of hydrogen-sulfide gas, an oxygen-rich condition, an oxygen-deficient condition, and the presence of carbon monoxide. The means for communicating the gas condition to a third party remote from the user includes a wireless communication device, and the wireless communication device includes at least one of a cellular radio, a local area network radio, and a personal area network radio.

Preferably, the means for communicating the gas condition to the third party remote from the user is configured to communicate the location of the device. In particular, in the preferred embodiment, the device is mobile. Also, the means for determining the location of the device may includes a global positioning system (GPS) receiver.

According to another embodiment, a method for using a device for notification of gas conditions is also disclosed herein. The method for using a device for notification of gas conditions preferably comprises the steps of: step one, detecting a gas condition; step two, communicating the gas condition to a user; step three, determining a location of the device; step four, recording the gas condition and the location of the device; and step five, communicating the gas condition and the location to a third party, the third party remote from the user.

For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and methods of use for the present disclosure, a system and device for notification of gas conditions, constructed and operative according to the teachings of the present disclosure.

FIG. 1 illustrates a device for notification of gas conditions during an ‘in-use’ condition, according to an embodiment of the disclosure.

FIG. 2 is a schematic diagram of a device for notification of gas conditions, according to an embodiment of the disclosure.

FIG. 3A is a perspective view illustrating a device for notification of gas conditions, according to an embodiment of the disclosure.

FIG. 3B is a rear view illustrating the device for notification of gas conditions of FIG. 3A, according to an embodiment of the disclosure.

FIG. 4 is a front view, illustrating the device for notification of gas conditions of FIG. 3B, according to an embodiment of the disclosure.

FIG. 5 is a flow diagram illustrating a method of use for device for notification of gas conditions according to an embodiment of the present disclosure.

The various embodiments of the present invention will hereinafter be described in conjunction with the appended drawings, wherein like designations denote like elements.

### DETAILED DESCRIPTION

As discussed above, embodiments of the present disclosure relate to a gas monitoring and more particularly to a device for notification of gas conditions as used to improve the detection and notification of the presence of potentially dangerous gas conditions.

The present invention may hold significant improvements and serves as a device for notification of gas conditions. Preferably, a gas monitoring device should provide a means for detecting the presence of potentially dangerous gas conditions and altering a user and a third party remote from the user to the presence and location of the gas conditions and, yet would operate reliably and be manufactured at a modest expense.

Generally speaking, a device for notification of gas conditions may communicate wirelessly (via cellular radio, a local area network radio, and/or a personal area network radio) to others onsite along with corporate offices to notify all essential personnel of a dangerous situation. The device may include three buttons on the exterior of the device such that a user can push the buttons in emergency situations (e.g., 1. For caution, 2. For dangerous, and 3. For catastrophic situations/conditions) to notify others.

The device for notification of gas conditions allows for immediate communication between all team members on site along with including corporate and safety management of emergency situations, emergency procedures, and the appropriate emergency response can be initiated along with providing global positioning system (GPS) coordinates for an exact location for emergency services (e.g., ambulance, fire, police, etc.).

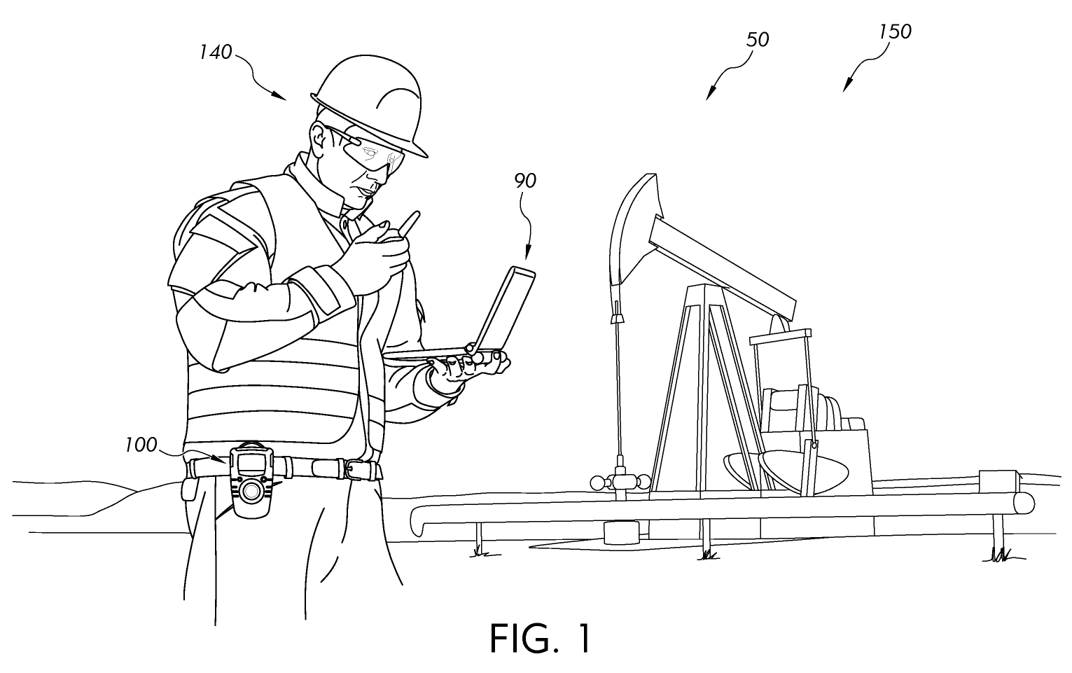

Referring now more specifically to the drawings by numerals of reference there is shown in FIGS. 1-4, various views of a wireless personal gas monitor detector, or device for notification of gas conditions 100 (hereinafter, also “device 100”). The device 100 is a device made to monitor and detect the presence of gases such as hydrogen sulfide gas. The device 100 may include benefits of various personal monitoring systems, along with the addition of other features, in unique combination.

FIG. 1 illustrates the device for notification of gas conditions 100 during an ‘in-use’ condition 150, according to an embodiment of the disclosure. Here, the device 100 provides the ability to alert the user 140 and other personnel (third parties remote from the user) of the unsafe environment. Other personnel may include those also on the jobsite 50, and safety management at a regional or corporate office, along with the authorities. The device 100 may transmit an alert using Wi-Fi, radio frequency, cellular, and Bluetooth applications. The device 100 may also include a simple user interface (e.g., one or more push buttons) that when pressed signal caution, dangerous, and catastrophic conditions, for example. The device 100 additionally may include location monitoring (e.g., GPS), providing precise location of the emergency (e.g., the jobsite, location within the jobsite, location within a structure/vessel, etc.), which can be relayed to first responders. In addition, the device 100 may provide for the information being transmitted to be stored and retained (e.g., locally, on another mobile device 90, on a cloud-based server, etc.) and used by companies to mitigate future hazards along with the immediate notifications onsite. The exact specifications may vary.

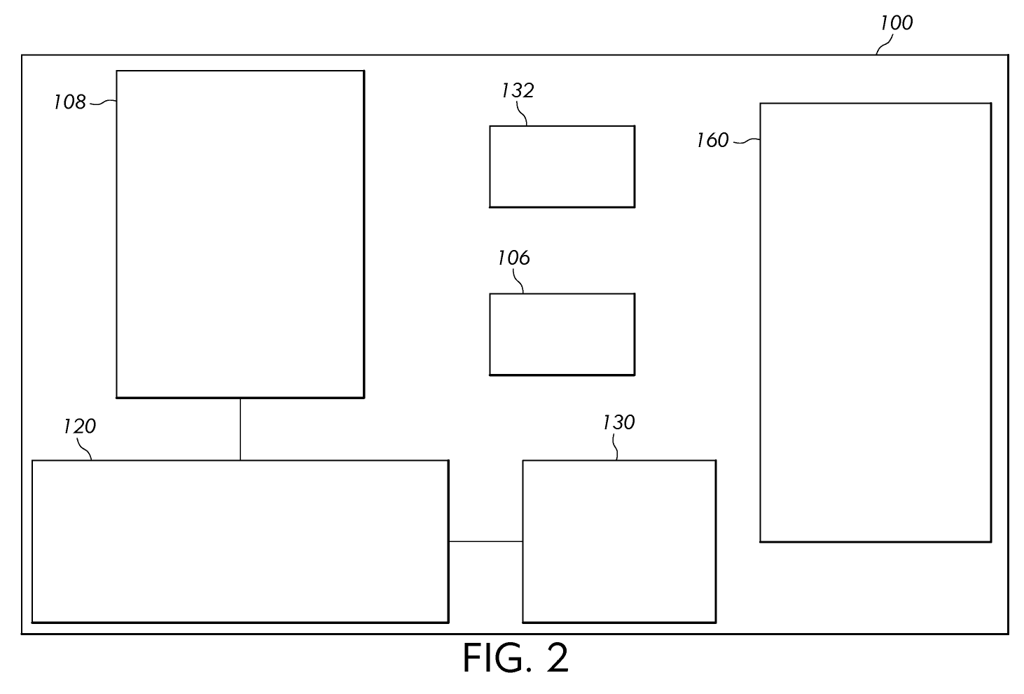

FIG. 2 is a schematic diagram of the device for notification of gas conditions, according to an embodiment of the disclosure. Here, the device for notification of gas conditions 100 may include (at least), means for detecting a gas condition 160, means for communicating gas condition to user 108, and means for communicating gas condition to a third party remote from user 120.

In some embodiments, the device 100 may be mobile and may further include means for determining a location of device 130. In particular, the means for determining the location of the device 130 may include a GPS receiver 122. As such, the device 100 may information including location monitoring (e.g., GPS). Further, the means for determining the location of the device 130 may be configured to provide precise location of the emergency (e.g., the jobsite in general, a location within the jobsite, a location within a structure/vessel, etc.), which can be relayed to first responders. According to one embodiment, the means for communicating the gas condition to a third party remote from the user 120 may be further configured to communicate the location of the device 100.

In other embodiments, the device 100 may further include a memory 132. In particular, the memory 132 may be configured for recording the gas condition and/or the location of device 100. In addition, the device 100 may provide for all or part of the information being transmitted to be stored and retained. Furthermore, the memory 132 may be local to the device 100, remotely located (e.g., cloud based), distributed across a plurality of devices/servers, or any combination thereof.

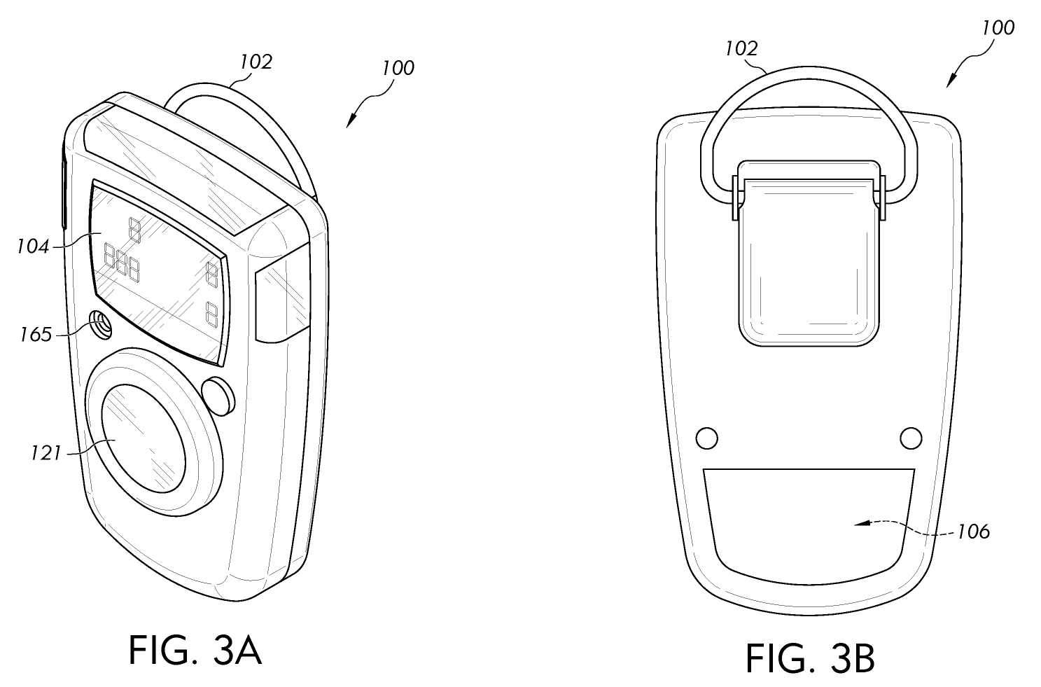

FIG. 3A is a perspective view illustrating the device for notification of gas conditions, according to an embodiment of the disclosure. Means for detecting a gas condition 160 may include a gas detector or gas sensor 165. The means for detecting the gas condition 160, may identify potentially hazardous gases by the use of one or more sensors represented by gas sensor 165. Such sensors can employ an audible alarm to alert people when a dangerous gas has been detected. It should be understood that a plurality of sensors may be deployed about the device 100 and may operate using different technologies. For example, some common types of sensors include infrared sensors, ultrasonic sensors, electro-chemical sensors, and semiconductor-based sensors.

The gas sensor 165 can be selected/configured to detect combustible, flammable and toxic gases, and oxygen rich and/or oxygen deficient condition. This type of detector is used widely in the petroleum (e.g., oil and gas, etc.) industry, and can be found in locations, such as on oil rigs, to monitor manufacturing or refinery processes. Gas detectors/sensors (and hence device 100) may also be configured for use in firefighting environments.

According to one embodiment, the gas sensor 165 may be configured to detect and/or measure hydrogen-sulfide gas. Also, the gas sensor 165 may include an oxygen sensor, or otherwise be further configured to detect and/or measure oxygen. In this configuration, the device 100 may determine oxygen-rich conditions and/or oxygen-deficient conditions with the gas sensor 165. Also, the means for detecting gas condition 160 may include a sensor configured to detect carbon monoxide 162. With these and other types of sensors, the device 100 can configured for use in many applications, such as in industrial plants, oil and gas refineries and/or extracting locations, as well as waste-water treatment facilities, vehicles, and/or homes/buildings.

FIG. 3B is a rear view illustrating the device for notification of gas conditions, according to an embodiment of the disclosure. According to one embodiment, the device 100 may further include at least one include an energy storage, such as rechargeable battery 106. Other embodiments may include removable and/or a replaceable battery/batteries.

According to one embodiment, device 100 may further include attachment fastener 102 configured to removably affix the device 100 to user 140. The fastener may be a closed loop, an openable loop (e.g., carabiner), lace, etc.

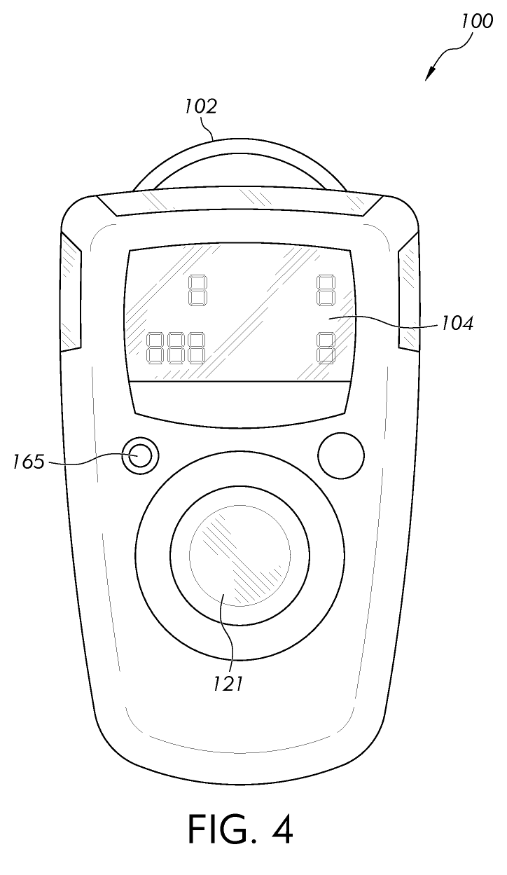

FIG. 4 is a front view, illustrating a device for notification of gas conditions, according to an embodiment of the disclosure. The means for communicating gas condition (e.g., a leak, etc.) to user 108 may be configured to communicate a plurality of severities of gas conditions to user. For example, the plurality of severities of gas condition may include a caution-level of severity, a danger-level of severity, and a catastrophic-level of severity 110.

Also, the means for communicating the gas condition to user 108 may include a display 104. The display 104 may be configured to visually communicate the gas condition to user 140, including, for example, the type of gas, the severity of the condition (as above), measurement information, and the like. The display 104 may also be configured to visually communicate operational and location information to the user, as well as information regarding the device 100. According to one embodiment, the means for communicating the gas condition to user 108 may include a speaker configured to aurally communicate the gas condition to use 140.

Returning to FIG. 1, the means for communicating the gas condition to user 108 may additionally be configured to communicate a plurality of gas conditions together or individually. This may similarly apply to the means for communicating the gas condition to a third party 120. For example, as above, the gas condition may include a presence of hydrogen-sulfide gas, an oxygen-rich condition, an oxygen-deficient condition, and/or the presence of carbon monoxide.

The means for communicating the gas condition to a third party remote from user 120 may include wireless communication device, where wireless communication device may include at least one of: cellular radio, local area network radio (LAN radio), and/or a personal area network radio (PAN radio). For example, the device 100 may transmit an alert using Wi-Fi, radio frequency, cellular, and/or Bluetooth applications.

The means for communicating the gas condition to a third party remote from user 120 may also include a simple user interface (e.g., one or more push buttons) that when pressed may signal caution, dangerous, and catastrophic conditions, for example. For example, the device may include a selectable report button 121 configured to allow the user 140 to select the gas condition/severity and send with the report button 121. Also for example, the device may include three buttons on the exterior of the device such that a user can push the buttons in emergency situations (e.g., 1. For caution, 2. For dangerous, and 3. For catastrophic situations/conditions) to notify others.

Device 100 for notification of gas conditions may be manufactured and provided for sale in a wide variety of sizes and shapes for a wide assortment of applications. Upon reading this specification, it should be appreciated that, under appropriate circumstances, considering such issues as design preference, user preferences, marketing preferences, cost, structural requirements, available materials, technological advances, etc., other kit contents or arrangements such as, for example, including more or less components, customized parts, different color combinations, parts may be sold separately, etc., may be sufficient.

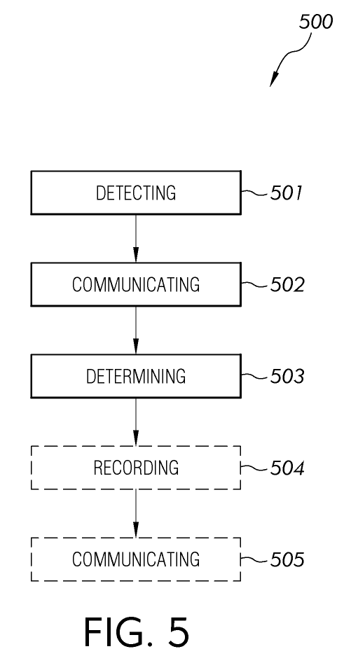

Referring now to FIG. 5 showing a flow diagram illustrating method of use 500 for device for notification of gas conditions 100, according to an embodiment of the present invention of FIGS. 1-4. As shown, method of use 500 may include the steps of: step one 501, detecting a gas condition; step two 502, communicating the gas condition to user; step three 503, determining a location of device 100; step four 504, recording the gas condition and the location of device; and step five 505, communicating the gas condition and the location to a third party, the third party being remote from user.

It should be noted that step five 505 and step six 506 are optional steps and may not be implemented in all cases. Optional steps of method of use 500 are illustrated using dotted lines in FIG. 5 so as to distinguish them from the other steps of method of use 500. It should be further noted that the steps described in the method 500 can be carried out in many different orders according to user preference. The use of “step of” should not be interpreted as “step for”, in the claims herein and is not intended to invoke the provisions of 35 U.S.C. §112(f).

The embodiments of the invention described herein are exemplary and numerous. Modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is new and desired to be protected by Letters Patent is set forth in the appended claims:

1. A device for notification of gas conditions, said device comprising:
means for detecting a gas condition;
means for communicating said gas condition to a user; and
means for communicating said gas condition to a third party remote from said user.

2. The device of claim 1, further comprising:
means for determining a location of said device; and
at least one rechargeable battery;
wherein said means for communicating said gas condition to said third party remote from said user is configured to communicate said location of said device; and
wherein said device is a mobile device.

3. The device of claim 2, wherein said means for determining said location of said device includes a GPS receiver.

4. The device of claim 2, further comprising a memory configured for recording said gas condition and said location of said device.

5. The device of claim 1, further comprising an attachment fastener configured to removably affix said device to said user.

6. The device of claim 1, wherein said means for detecting said gas condition includes a sensor configured to detect hydrogen-sulfide gas.

7. The device of claim 1, wherein said means for detecting said gas condition includes an oxygen sensor.

8. The device of claim 7, wherein said gas condition includes an oxygen-rich condition.

9. The device of claim 7, wherein said gas condition includes an oxygen-deficient condition.

10. The device of claim 1, wherein said means for detecting said gas condition includes a sensor configured to detect carbon monoxide.

11. The device of claim 1, wherein said means for communicating said gas condition to said user is configured to communicate a plurality of severities of said gas condition to said user.

12. The device of claim 11, wherein said plurality of severities of said gas condition include a caution-level of severity, a danger-level of severity, and a catastrophic-level of severity.

13. The device of claim 1, wherein said means for communicating said gas condition to said user includes a display configured to visually communicate said gas condition to said user.

14. The device of claim 1, wherein said means for communicating said gas condition to said user includes a speaker configured to aurally communicate said gas condition to said user.

15. The device of claim 1, wherein said means for communicating said gas condition to said user is configured to communicate a plurality of gas conditions including a presence of hydrogen-sulfide gas, an oxygen-rich condition, an oxygen-deficient condition, and a presence of carbon monoxide.

16. The device of claim 1, wherein said means for communicating said gas condition to said third party remote from said user includes a wireless communication device.

17. The device of claim 1, wherein said wireless communication device includes at least one of a cellular radio, a local area network radio, and a personal area network radio.

18. A device for notification of gas conditions, said device comprising:
means for detecting a gas condition;
means for communicating said gas condition to a user;
means for communicating said gas condition to a third party remote from said user;
means for determining a location of said device;
at least one rechargeable battery;
a memory configured for recording said gas condition and said location of said device; and
an attachment fastener configured to removably affix said device to said user;
wherein said means for communicating said gas condition to said third party remote from said user is configured to communicate said location of said device;
wherein said device is a mobile device;
wherein said means for determining said location of said device includes a GPS receiver;
wherein said means for detecting said gas condition includes a sensor configured to detect hydrogen-sulfide gas;
wherein said means for detecting said gas condition includes an oxygen sensor;
wherein said gas condition includes an oxygen-rich condition;
wherein said gas condition includes an oxygen-deficient condition;
wherein said means for detecting said gas condition includes a sensor configured to detect carbon monoxide;
wherein said means for communicating said gas condition to said user is configured to communicate to a plurality of severities of said gas condition to said user;
wherein said plurality of severities of said gas condition include a caution-level of severity, a danger-level of severity, and a catastrophic-level of severity, said plurality of severities of said gas condition manually alertable by said user;
wherein said means for communicating said gas condition to said user includes a display configured to visually communicate said gas condition to said user;
wherein said means for communicating said gas condition to said user includes a speaker configured to aurally communicate said gas condition to said user;
wherein said means for communicating said gas condition to said user is configured to communicate to a plurality of gas conditions including a presence of hydrogen-sulfide gas, an oxygen-rich condition, an oxygen-deficient condition, and a presence of carbon monoxide;
wherein said means for communicating said gas condition to a third party remote from said user includes a wireless communication device; and
wherein said wireless communication device includes at least one of a cellular radio, a local area network radio, and a personal area network radio.

19. A method for notification of gas conditions, said method comprising:
detecting a gas condition;
communicating said gas condition to a user; and
determining a location of a device.

20. The method of claim 19, further comprising the steps of:
recording said gas condition and said location of said device, and
communicating said gas condition and said location to a third party, said third party remote from said user.
